(19) United States
(12) Patent Application Publication
(10) Pub. No.: US 2022/0236208 A1
(43) Pub. Date: Jul. 28, 2022

(54) SYSTEM FOR BLOOD GLUCOSE METER COUPLED WITH MOBILE ELECTRONIC DEVICE

(71) Applicant: Roche Diabetes Care, Inc., Indianapolis, IN (US)

(72) Inventor: Scott E. Carpenter, Pendleton, IN (US)

(73) Assignee: Roche Diabetes Care, Inc., Indianapolis, IN (US)

(21) Appl. No.: 17/718,491

(22) Filed: Apr. 12, 2022

Related U.S. Application Data
(63) Continuation of application No. PCT/US2020/056007, filed on Oct. 16, 2020.
(60) Provisional application No. 62/916,817, filed on Oct. 18, 2019.

(51) Int. Cl. G01N 27/327; H04W 4/80; H04B 5/00; H04B 5/02; G06F 21/57; H04L 9/32; G01N 33/66

(52) U.S. Cl. CPC G01N 27/3273; H04W 4/80; H04B 5/0081; G06F 2221/033; G06F 21/572; H04L 9/3239; G01N 33/66; H04B 5/02

(57) ABSTRACT

A hybrid analyte test meter includes a processor operatively connected to a memory, measurement signal generator, measurement signal receiver, and short range wireless transceiver. The processor executes firmware instructions in the memory to operate the measurement signal generator to apply electrical signals to a sample deposited on the electrochemical test strip via the port, receive signal measurements from the measurement signal receiver in response to the predetermined sequence of electrical signals, and transmit data corresponding to the plurality of signal measurements to an external computing device using the short range wireless transceiver, wherein the processor does not identify a measurement of an analyte in the sample based on the plurality of signal measurements.

### CLAIM OF PRIORITY

This application claims the benefit of International Application No. PCT/US2020/056007, which is entitled “SYSTEM FOR BLOOD GLUCOSE METER COUPLED WITH MOBILE ELECTRONIC DEVICE,” and was filed on 16 Oct. 2020, the entire contents of which are incorporated herein by reference. This application claims the further benefit of U.S. Provisional Application No. 62/916,817, which is entitled “SYSTEM FOR BLOOD GLUCOSE METER COUPLED WITH MOBILE ELECTRONIC DEVICE,” and was filed on 18 Oct. 2019, the entire contents of which are incorporated herein by reference.

### TECHNICAL FIELD

The disclosure relates generally to the field of analyte detection in fluid samples and, more specifically, to devices that detect analytes in fluid samples including blood glucose meters.

### BACKGROUND

Analyte test meters that are known to the art enable the analysis of a bodily fluid sample provided by a user to identify the level of one or more analytes in the body of the user using an electronic device and one or more electrochemical reactions. These analyte meters provide significant benefits for the accurate measurement of analytes in fluidic samples (i.e., biological or environmental) for individual users. An analyte meter applies electrical signals to the combination of the reagents and the fluid sample and records responses to the applied electrical signals, and a combination of electronic hardware and software in the analyte test meter implements a detection engine that detects a level of the analyte in the body of the user based on the recorded responses to the electrical signals. For example, persons with diabetes can benefit from measuring glucose by providing a fluid sample of blood or another bodily fluid to reagents that are formed on an electrochemical test strip, which is electrically connected to a blood glucose meter (BGM). The BGM provides a measurement of the blood glucose level of the user, and many BGM devices use single-use electrochemical test strips that are discarded after each blood glucose measurement. Analyte test meters can also provide benefits to users at-risk for heart disease by providing measurements of cholesterols and triglycerides, among other analytes. These are but a few examples of the benefits of measuring analytes in biological samples. Advancements in the medical sciences are identifying a growing number of analytes that can be electrochemically analyzed in a fluidic sample.

While analyte test meters that are known to the art can provide measurements for a wide range of analytes, existing analyte devices are often incapable of receiving software and firmware updates during the lifetime of the analyte device, which is often several years in common practice. While the analyte test meter remains functional even without updates, the inability to update the analyte test meter may result in inefficient operation. For example, as described above analyte test meters use test strips, which are typically disposable after a single use. Over the life of the meter, the structure and chemistry of the test strips cannot change in any appreciable way because any such change would likely reduce the accuracy of the existing analyte test meters. Even comparatively small manufacturing variations that can occur between different batches of test strips can negatively impact the accuracy with existing analyte test meters that cannot be dynamically updated to provide accurate measurements from different test strips even if the test strips themselves are not defective. Many analyte test meters operate as self-contained devices that cannot receive updates and in particular low-cost analyte test meters are often incapable of receiving firmware updates. While some analyte test meters that incorporate network connectivity have the technical capability to receive updated firmware data from an online update service, these meters often do not receive updates as a practical matter because the same capability that enables legitimate firmware updates also enables unauthorized firmware updates that cause the meter to operate in a manner that is not authorized by the FDA or by other health regulation authorities.

Another challenge that confronts existing analyte test meters is that these test meters generally operate as standalone devices with integrated microprocessors, input devices, and output devices, and in some instances network devices, which increase the complexity, price, and power consumption of the analyte test meter. Even analyte test meters that are configured to transmit results to external computing devices via, for example, a Bluetooth or other wireless data link are still configured to act as standalone devices during normal operation. While some removable analyte test meters are configured to act as dependent devices that connect to another host device, such as a personal computer (PC), smartphone, or other digital device via a universal serial bus (USB) or similar connection, these removable analyte test meters still implement the full hardware requirements to implement the analyte measurement process and cannot receive updates in the field. Furthermore, these removable analyte test meters generally interfere with the normal operation of a digital device if they remain plugged into the device, and therefore a user must connect and disconnect the removable analyte test meter to and from the host device prior for each use. Consequently, improvements to analyte test meters that address the challenges described above would be beneficial.

### SUMMARY

In one embodiment, a hybrid analyte test meter has been developed. The hybrid analyte test meter includes a memory configured to store firmware instructions, a port configured to receive an electrochemical test strip, a measurement signal generator electrically connected to the port, a measurement signal receiver electrically connected to the port, a short range wireless transceiver, and a processor operatively connected to the memory, the measurement signal generator, the measurement signal receiver, and the short range wireless transceiver. The processor is configured to execute the firmware instructions in the memory to operate the measurement signal generator to apply a predetermined sequence of electrical signals to a sample deposited on the electrochemical test strip via the port, receive a plurality of signal measurements from the measurement signal receiver, the measurement signal receiver generating the plurality of measured signals based on a plurality of electrical signals received from the electrochemical test strip in the port in response to the predetermined sequence of electrical signals, and transmit data corresponding to the plurality of signal measurements to an external computing device using the short range wireless transceiver, wherein the data corresponding to the plurality of signal measurements enable another processor in the external computing device to identify a measurement of an analyte in the sample.

In another embodiment, an analyte test meter has been developed. The analyte test meter includes a hybrid analyte test meter and a mobile electronic device. The hybrid analyte test meter includes a first memory configured to store firmware instructions, a port configured to receive an electrochemical test strip, a measurement signal generator electrically connected to the port, a measurement signal receiver electrically connected to the port, a first short range wireless transceiver, and a first processor operatively connected to the first memory, the measurement signal generator, the measurement signal receiver, and the first short range wireless transceiver. The first processor is configured to execute the firmware instructions in the first memory to operate the measurement signal generator to apply a predetermined sequence of electrical signals to a sample deposited on the electrochemical test strip via the port, receive a plurality of signal measurements from the measurement signal receiver, the measurement signal receiver generating the plurality of signal measurements based on a plurality of electrical signals received from the electrochemical test strip in the port in response to the predetermined sequence of electrical signals, and transmit data corresponding to the plurality of signal measurements to the mobile electronic device using the first short range wireless transceiver. The mobile electronic device includes a second memory configured to store software instructions, a second short range wireless transceiver, an output device, and a second processor operatively connected to the second memory, the second short range wireless transceiver, and the output device. The second processor is configured to execute the software instructions in the second memory to receive the plurality of signal measurements from the hybrid analyte test meter using the second short range wireless transceiver, execute an analyte detection algorithm to identify a level of the analyte in the sample based on the plurality of signal measurements, and generate an output with the output device to present the level of the analyte in the sample to a user.

### BRIEF DESCRIPTION OF THE DRAWINGS

The advantages, effects, features and objects other than those set forth above will become more readily apparent when consideration is given to the detailed description below. Such detailed description makes reference to the following drawings, wherein:

FIG. 1 is a set of views of an analyte test meter that includes a hybrid analyte test meter and an external computing device that is embodied as a mobile electronic device.

FIG. 2 is a schematic diagram of the analyte test meter of FIG. 1 in a system that provides networked communication services to the analyte test meter.

FIG. 3 is a block diagram of a process for operation of the analyte test meter of FIG. 1 and the system of FIG. 2.

FIG. 4 is a block diagram of a process for updating software and firmware in the analyte test meter of FIG. 1 and the system of FIG. 2.

### DETAILED DESCRIPTION

These and other advantages, effects, features and objects are better understood from the following description. In the description, reference is made to the accompanying drawings, which form a part hereof and in which there is shown by way of illustration, not limitation, embodiments of the inventive concept. Corresponding reference numbers indicate corresponding parts throughout the several views of the drawings.

While the inventive concept is susceptible to various modifications and alternative forms, exemplary embodiments thereof are shown by way of example in the drawings and are herein described in detail. It should be understood, however, that the description of exemplary embodiments that follows is not intended to limit the inventive concept to the particular forms disclosed, but on the contrary, the intention is to cover all advantages, effects, and features falling within the spirit and scope thereof as defined by the embodiments described herein and the claims below. Reference should therefore be made to the embodiments described herein and claims below for interpreting the scope of the inventive concept. As such, it should be noted that the embodiments described herein may have advantages, effects, and features useful in solving other problems.

The devices, systems and methods now will be described more fully hereinafter with reference to the accompanying drawings, in which some, but not all embodiments of the inventive concept are shown. Indeed, the devices, systems and methods may be embodied in many different forms and should not be construed as limited to the embodiments set forth herein; rather, these embodiments are provided so that this disclosure will satisfy applicable legal requirements.

Likewise, many modifications and other embodiments of the devices, systems and methods described herein will come to mind to one of skill in the art to which the disclosure pertains having the benefit of the teachings presented in the foregoing descriptions and the associated drawings. Therefore, it is to be understood that the devices, systems and methods are not to be limited to the specific embodiments disclosed and that modifications and other embodiments are intended to be included within the scope of the claims. Although specific terms are employed herein, they are used in a generic and descriptive sense only and not for purposes of limitation.

Unless defined otherwise, all technical and scientific terms used herein have the same meaning as commonly understood by one of skill in the art to which the disclosure pertains. Although any methods and materials similar to or equivalent to those described herein can be used in the practice or testing of the methods, the preferred methods and materials are described herein.

Moreover, reference to an element by the indefinite article “a” or “an” does not exclude the possibility that more than one element is present, unless the context clearly requires that there be one and only one element. The indefinite article “a” or “an” thus usually means “at least one.” Likewise, the terms “have,” “comprise” or “include” or any arbitrary grammatical variations thereof are used in a non-exclusive way. Thus, these terms may both refer to a situation in which, besides the feature introduced by these terms, no further features are present in the entity described in this context and to a situation in which one or more further features are present. For example, the expressions “A has B,” “A comprises B” and “A includes B” may refer both to a situation in which, besides B, no other element is present in A (i.e., a situation in which A solely and exclusively consists of B) or to a situation in which, besides B, one or more further elements are present in A, such as element C, elements C and D, or even further elements.

As used herein, the term “mobile electronic device” refers to a portable computing device that provides a user one or more of each of the following components: an output device, an input device, a memory, and a wireless communication device that are controlled by one or more processors in the mobile electronic device. Examples of output devices include, but are not limited to, liquid crystal display (LCD) displays, organic or inorganic light emitting diode (LED) displays, and other forms of graphical display device, audio speakers, and haptic feedback devices. Examples of input devices include, but are not limited to buttons, keyboards, touchscreens, and audio microphones. Examples of memory include, but are not limited to, both volatile data storage devices such as random-access memory (RAM) and non-volatile data storage devices such as magnetic disks, optical disks, and solid-state storage devices including EEPROMs, NAND flash, or other forms of solid-state data storage devices. Examples of wireless communication devices include, but are not limited to, radio transceivers that operate with the Near Field Communication (NFC) protocol, the Bluetooth protocol family, including Bluetooth Low Energy (BLE), the IEEE 802.11 protocol family (“Wi-Fi”), and cellular data transmission standards (“4G,” “5G,” or the like). Examples of the processors include digital logic devices that implement one or more central processing units (CPUs), graphics processing units (GPUs), digital signal processors (DSPs), field programmable gate arrays (FPGAs), application specific integrated circuits (ASICs), and any other suitable digital logic devices in an integrated device or as a combination of devices that operate together to implement the processor. Common examples of mobile electronic devices include, but are not limited to, smartphones, smart watches, and tablet computing devices.

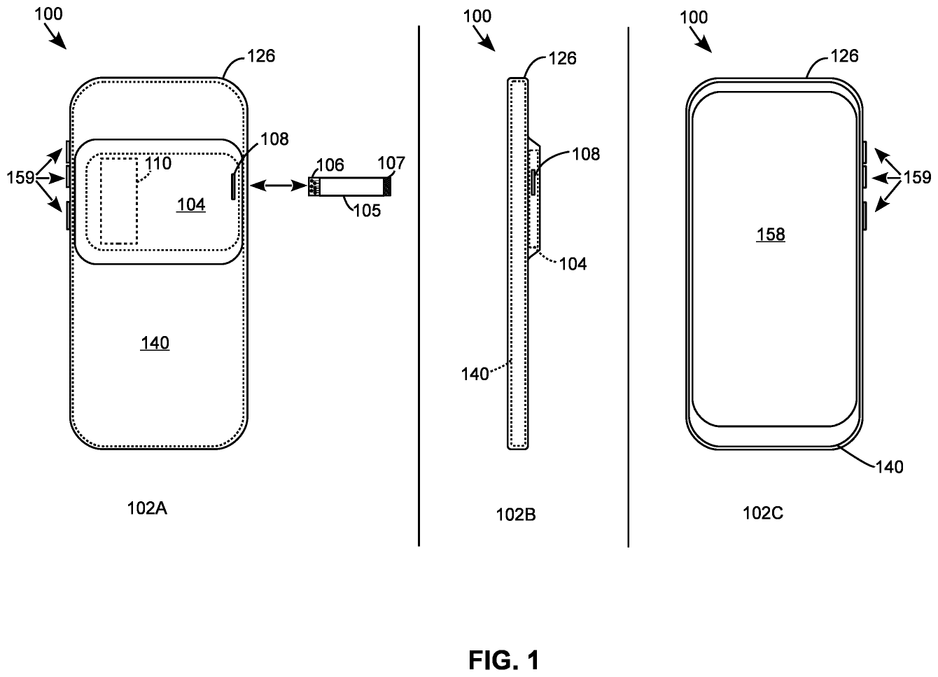

FIG. 1 depicts a rear view 102A, profile view 102B, and front view 102C of an analyte test meter 100. The analyte test meter 100 is formed from a hybrid analyte test meter 104 and a mobile electronic device 140 that interoperate as described herein to provide the function of the analyte test meter 100. As used herein, the terms “hybrid analyte test meter,” and “hybrid meter” are used interchangeably and refer to an analyte testing device that implements specific hardware and software elements to generate and apply electrical signals to a sample that includes the analyte and to record electrical responses to the electrical signals in an amperometric testing process. Unlike a conventional analyte test meter, however, the hybrid analyte test meter does not implement the hardware and software components that perform the full analyte measurement process and provide an output of the analyte measurement. Instead, the hybrid meter transmits digital data corresponding to the recorded electrical responses to the electrochemical testing sequence to an external computing device, which is embodied as the mobile electronic device 140 in FIG. 1. The mobile electronic device 140 is configured with hardware and software components that process the digital data received from the hybrid meter to generate a measurement of the analyte level in the sample, provide an output to a user of the measured analyte level, and to provide additional services to the user via the mobile electronic device 140 and optionally other computing devices that communicate with the mobile electronic device 140 via one or more data networks. As described below, the combination of the hybrid meter 104 with the mobile electronic device 140 or another external computing device to form the analyte measurement device 100 provides improvements to the efficiency and functionality of the hardware and software elements that form the analyte measurement device 100.

As depicted in view 102A, the hybrid analyte test meter 104 is mounted to a rear surface of the mobile electronic device 140, which is embodied as a smartphone in FIG. 1. A case 126 encloses both the hybrid meter 104 and the mobile electronic device 140 to hold the hybrid meter 104 in a fixed position relative to the mobile electronic device 140. The hybrid meter 104 includes a port 108 that accepts a removable electrochemical test strip 105. The electrochemical test strip 105 includes electrical contacts 106 that form an electrical connection with the hybrid meter 104. The electrical contacts 106 are electrically connected to electrodes in a sample region 107 that supports a chemical reagent in contact with the electrodes. As is generally known in the art, the reagent includes an enzyme and a mediator that support a reduction-oxidation (redox) reaction with an analyte in a fluid sample that is applied to the reagent, and the hybrid meter 104 applies electrical potentials to the test strip to detect electrical currents with current levels that are affected by these redox reactions. The port 108 includes a physical opening in a housing of the hybrid meter 104 to accept a portion of the electrochemical test strip 105 that includes the electrical connectors 106. During operation, the electrical contacts 106 of the electrochemical test strip 105 are inserted in the port 108 while the sample region 107 remains outside of the hybrid meter 104 and extends beyond the housing of the mobile electronic device 140 to enable a user to apply a dose of a fluid sample, such as blood, to the reagent in the sample region 107. The hybrid meter 104 applies a test sequence of electrical signals to the electrical contacts 106 and measures electrical signal responses, which the hybrid meter 104 converts to digital data and transmits to the mobile electronic device 140. The electrochemical test strip 105 is representative of electrochemical test strips that are otherwise known to the art, and the test meter 100 is operable using various forms of electrochemical test strip that are known to the art without requiring modification of the electrochemical test strips.

The hybrid meter 104 includes an optional storage compartment 110 that stores consumable supplies that are used with the test meter 100 including, for example, a supply of one or more of the electrochemical test strips. Other consumable components include, for example, lancing needles that enable a user to produce a blood sample to dose a test strip. In some embodiments, the storage compartment 110 has a moisture-resistant door and stores a desiccant that prevents contamination of stored test strips with water prior use of the electrochemical test strips.

View 102B in FIG. 1 depicts a profile view of the hybrid meter 104 and the mobile electronic device 140 of the meter 100. As depicted in the profile view 102B, the hybrid meter 104 extends from the rear surface of the mobile electronic device 140. The hybrid meter 104 is formed with a thickness of 10 mm or less and, in one embodiment, a thickness of approximately 5 mm to enable a user to hold and carry the combination of the mobile electronic device 140 and the hybrid meter 104 in a convenient manner. Additionally, the hybrid meter 104 is formed with the general shape of a rectangular prism with a lower base that is proximate to the mobile electronic device 140 and an upper base that engages the case 126. The case 126 provides sloped edges with rounded corners surrounding the hybrid meter 104 to protect the hybrid meter 104. However, those of skill in the art will recognize that alternative configurations of the hybrid analyte test meter 104 may have a different thicknesses or shapes that enable engagements with different types of mobile electronic devices.

View 102C in FIG. 1 depicts a front view of the mobile electronic device 140 in the meter 100. The case 126 surrounds the housing of the mobile electronic device 140 and enables full access to a display 158 and to mechanical interface buttons 159. The display 158 provides a graphical user interface to functions of the mobile electronic device 140, which include a software application that provides an interface for the user to operate the meter 100 in addition to standard functions that are implemented by the mobile electronic device 140. As depicted above the case 126 and the hybrid meter 104 do not interfere with user access to the display 158 or to a wired cable connection, such as a USB connection or other wired connection, in the mobile electronic device 140. More generally, the hybrid meter 104 provides minimal interference with the operation of the mobile electronic device 140 for uses outside of performing analyte measurement operations for the user while the entire meter device 100 also integrates the hybrid meter 104 so that the hybrid meter 104 remains attached to the mobile electronic device 140 during normal use and is available for use at any time the user accesses the mobile electronic device 140.

As depicted in FIG. 1, the case 126 secures the hybrid analyte test meter 104 to the mobile electronic device 140. The case 126 encloses at least a portion of the hybrid meter 104 and at least a portion of the mobile electronic device 140. In the embodiment of FIG. 1, the case 126 includes a first cavity that contains the hybrid analyte test meter 104 and a second cavity that contains the mobile electronic device 140. In this embodiment, the case 126 is formed from rubber, plastic, or another flexible material that holds the hybrid meter 104 in place proximate to a rear surface of the mobile electronic device 140 and wraps around the edges of the mobile electronic device 140 while providing openings for the display 158 and interface buttons 159. While not shown in greater detail, the case 126 can provide additional openings for camera lenses, receptacles for wired connections such as USB ports, or other components in the mobile electronic device 140. The case 126 also provides an opening for the electrochemical test strip port 108 in the hybrid meter 104 and, if needed, an opening to provide access to the storage compartment 110. The case 126 holds the hybrid meter 104 in a fixed position with the mobile electronic device 140 to enable a user to hold and operate both devices as a single unit. Of course, since different mobile electronic devices have a variety of shapes and sizes, different case designs can be used to hold the hybrid meter 104 with a wide range of mobile electronic devices without modification to the hybrid meter 104. The hybrid meter 104 is transferrable to a different case if, for example, a user obtains a different smartphone or other mobile electronic device for use with the hybrid meter 104. The case 126 also provides some degree of damage protection to both the hybrid meter 104 and the mobile electronic device 140, such as providing some damage protection against drops. In other embodiments, the hybrid meter 104 is attached to an exterior of a mobile electronic device using an adhesive coupling, a magnetic coupling, or a mechanical connection.

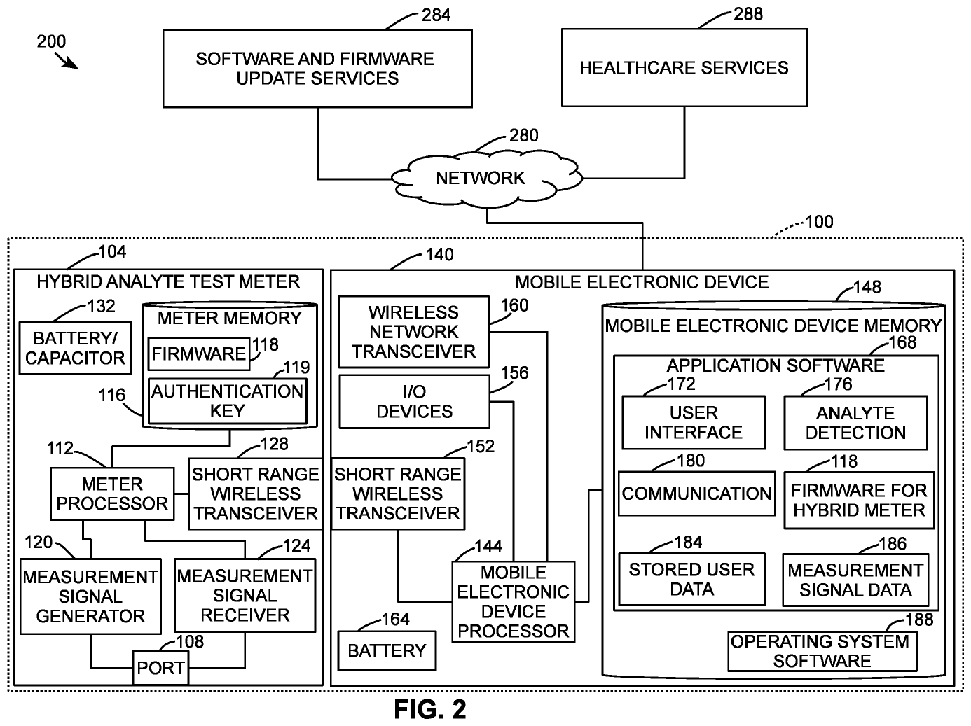

FIG. 2 is a schematic diagram of a system 200 that includes the analyte test meter 100 that utilizes a data network 280 to communicate with networked servers that provide software and firmware update services 284 and healthcare services 288. As described above, the analyte test meter 100 combines the hybrid meter 104 and the mobile electronic device 140. In the system 200, the software and firmware update services are commercially available services such as so-called “app stores” or other online services that provide mechanisms to update the software in the mobile electronic device 140 and, in the specific configuration of FIG. 2, the firmware of the hybrid meter 104 via the mobile electronic device 140. The healthcare services 288 represents an online system that receives analyte measurements and other user data from the analyte test meter 100. The healthcare services 288 optionally provides health information and treatment advice to the user of the analyte test meter 100, and in some embodiments the healthcare services 288 also enables healthcare providers (HCPs) to access a history of analyte levels for the user as part of providing healthcare services to the user. FIG. 2 further depicts the internal components and configuration of the analyte test meter 100 in more detail.

As depicted in FIG. 2, the hybrid meter 104 includes a first processor 112 that is operatively connected to a first memory 116, a first short range wireless transceiver 128, and to the port 108 via a measurement signal generator 120, and a measurement signal receiver 124. A battery or capacitor 132 provides electrical power to operate these components in the hybrid meter 104. The mobile electronic device 140 includes a second processor 144 that is operatively connected to a second memory 148, a second short range wireless transceiver 152, input/output (1/0) devices 156, and a wireless network transceiver 160. During operation, the processor 144 in the mobile electronic device 140 executes application software 168 to provide an interface to a user of the analyte test meter 100, to control the hybrid meter 104, and to analyze measurement data that are received from the hybrid meter 104 to identify a level of one or more analytes in a sample on an electrochemical test strip that is provided to the hybrid meter 104. A battery 164 provides electrical power to operate these components in the mobile electronic device 140.

Referring to the hybrid analyte test meter 104 in more detail, the memory 116 includes a non-volatile memory device such as an EEPROM, NAND, or other suitable data storage device that holds firmware data 118 and a firmware authentication key 119 in long-term storage. The memory 116 further includes a volatile RAM that stores data such as recorded signal measurement data and any other data that are generated and stored in the memory 116 during operation of the hybrid meter 104. The firmware 118 is embodied as binary data that include both operating instructions to control the operation of the processor 112 and parameter data that the processor 112 uses to control the operation of the measurement signal generator 120 and the measurement signal receiver 124. For example, the processor 112 executes instructions in the firmware 118 to operate the measurement signal generator 120, and the processor 112 uses parameters in the firmware 118 specify operating voltage levels and durations for AC and DC signals that the measurement signal generator 120 applies to the electrodes of a test strip via the port 108. Similarly, the processor 112 executes instructions in the firmware 118 to process and record analog or digitized signal measurement data that the measurement signal receiver 124 receives from the test strip in the port 108. The processor 112 also executes firmware instructions to perform communication with the mobile electronic device 140 using the short range wireless transceiver 128. As described in further detail below, the hybrid meter 104 receives updated firmware that the mobile electronic device 140 receives as part of a software update. The processor 112 uses the authentication key 119, which in one embodiment is a cryptographic public key of a trusted publisher, to verify the authenticity of an updated firmware image prior to using the updated firmware.

The measurement signal generator 120 includes modulators, amplifiers, smoothing filters, and other circuits to implement a waveform generator that is configurable to generate both direct current (DC) and alternating current (AC) signals within a predetermined operational range for voltages, power, and frequency. For example, in one configuration the measurement signal generator 120 can produce AC and DC voltages with a relative potential difference of up to 1.0 V (e.g. +0.5 V to −0.5 V) between the counter electrode and the reference electrode in a test strip at frequencies of 0 Hz (DC) up to 100 kHz AC with varying waveforms including sinusoidal and triangular AC waveforms and square or trapezoidal pulsed DC waveforms. A digital-to-analog converter (DAC) that is integrated in the processor 112 or in the measurement signal generator enables the processor 112 to generate a digital data output that produces various analog voltage measurement signals from the measurement signal generator 120. The measurement signal receiver 124 includes one or more signal amplifiers and filters that enable the detection of electrical current signals that are generated between the counter and reference electrodes in the test strip 105 in response to the measurement signals from the measurement signal generator 120. An analog-to-digital signal converter that is integrated in the processor 112 or in the measurement signal generator 120 enables the hybrid analyte test meter 104 to generate discrete digital sampling values of the measured current for further processing by digital logic devices in the hybrid meter 104 and the mobile electronic device 140. In some embodiments, either or both of the measurement signal generator 120 and the measurement signal receiver 124 are either wholly or partially integrated with the processor 112. For example, the processor 112 optionally integrates components such as the DACs and ADCs, modulators, amplifiers, and filter circuits. In other embodiments, the measurement signal generator 120 and the measurement signal receiver 124 are implemented using external components in which the processor 112 generates control signals to operate the signal generator 120 and the processor 112 receives signal measurement data from the measurement signal receiver 124.

In the embodiment of FIG. 2, the short range wireless transceiver 128 includes at least one antenna and at least one device that provides short range wireless communication with the mobile electronic device 140. In some embodiments, the short range wireless transceiver 128 further includes circuits that enable the mobile electronic device 140 to provide electrical power to the hybrid meter 104 via inductive coupling without requiring a wired electrical connection between the two devices. In one embodiment, the short range wireless transceiver 128 includes a near field communication (NFC) wireless transceiver that is connected to a coil antenna formed in the hybrid analyte test meter 104 and configured to receive digital data from the processor 112 for transmission to the mobile electronic device 140 and to receive and decode transmissions from the mobile electronic device 140 to provide digital data representation of the received signals to the processor 112. The hybrid meter 104 incorporates the coil antenna as a conductive trace that is formed in a printed circuit board or employs other electrically conductive coil in the hybrid analyte test meter 104. The antenna receives data from the mobile electronic device 140 that are encoded in electromagnetic signals that the corresponding short range wireless transceiver 152 in the mobile electronic device 140 emits and the receiver in the short range wireless transceiver 128 decodes the data for use by the processor 112.

In the embodiment of FIG. 2, the short range wireless transceiver 128 incorporates an NFC transceiver that provides an energy efficient wireless communication channel with a corresponding NFC transceiver in the short range wireless transceiver 152 of the mobile electronic device 140. The NFC transceivers operate over short distances (typically on the order of 5 cm or less) and the physical configuration of the analyte test meter 100 that places the hybrid analyte test meter 104 in close proximity to the mobile electronic device 140 enables effective use of the NFC transceivers to provide communications. Additionally, many mobile electronic devices also include other wireless transceivers such as IEEE 802.11 “Wi-Fi”, Bluetooth, and cellular data (e.g. 4G, 5G, etc.), which are not substantially affected by the use of the short range wireless transceiver, such as the NFC transceivers, which enables the mobile electronic device 140 to operate for general use outside of communication with the hybrid meter without interference from the hybrid meter 104. The short range wireless transceivers 128 and 152 typically operate with lower electrical power levels than other wireless network standards such as Bluetooth or IEEE 802.11 (“Wi-Fi”). Furthermore, because the case 126 holds the hybrid analyte test meter 104 in close proximity to the mobile electronic communication device 140 to enable inductive coupling between the coil antennas in both devices, the short range wireless transceivers 128 and 152 can communicate with each other with minimal interference from external radio transmitters, which avoids connectivity issues that are known to affect longer-range wireless transmission protocols in an environment with a large number of transmitting devices. Other embodiments of the short range wireless transceiver use radio frequency identification (RFID) transceivers or similar short range wireless technologies that do not interfere with the operation of additional wireless network transceivers in the mobile electronic device 140.

As described above, the short range wireless transceivers 128 and 152 provide wireless data communication between the hybrid meter 104 and the mobile electronic device 140. Additionally, some embodiments of the hybrid meter 104 use the short range wireless transceiver 128 to receive electrical power from the mobile electronic device 140 that charges a capacitor 132 or recharges a battery 132 to provide electrical power to components in the hybrid meter 104. In one embodiment that incorporates NFC transceivers, the mobile electronic device 140 transmits a power signal that provides electrical power to the NFC transceiver in the short range wireless transceiver 128, which then provides the electrical power to charge a capacitor 132 or to recharge a battery 132. As known in the art, the NFC power signal is transmitted as an alternating current (AC) signal at a predetermined frequency (e.g. 13.56 MHz), and the coil antennas in both the hybrid meter 104 and the mobile electronic device 140 enable inductive coupling to generate electrical power in the hybrid meter 104. The hybrid meter 104 includes a rectifier that converts the AC power signal to a direct current (DC) charging current to provide electrical power to a capacitor or a rechargeable battery 132. While some NFC transceiver configurations can implement the power transfer operations described above, other embodiments employ different charging circuits that provide inductive coupling between the coil antennas of the hybrid meter 104 and the mobile electronic device 140 that may use a different AC power signal frequency (e.g. 50 Hz or 60 Hz). As described above, the embodiments that provide a wireless power transfer from the mobile electronic device 140 to the hybrid meter 104 are optional, and other embodiments of the hybrid meter 104 employ a commercially-available non-rechargeable battery, such as a coin cell or other suitable battery, to provide electrical power.

The battery or capacitor 132 stores electrical energy that provides electrical power to operate the hybrid analyte test meter 104 including, more specifically, the processor 112, the memory 116, the measurement signal generator 120, the measurement signal receiver 124, and the short range wireless transceiver 128. In an embodiment that uses a battery 132, the hybrid analyte test meter 104 can be activated at any time. For example, the hybrid analyte test meter 104 can be activated via an electrical switch, such as a switch that is closed when the port 108 receives a test strip, or via a wireless activation signal that is received from the mobile electronic device 140. In an embodiment that uses a capacitor 132, the capacitor 132 only holds charge for a comparatively short time period (e.g. on the order of several minutes) and the hybrid analyte test meter 104 is activated in response to an external charging signal that the mobile electronic device 140 generates to charge the capacitor 132 via an inductive coupling through the short range wireless transceiver. Once the capacitor 132 reaches a predetermined charge level, the capacitor 132 provides the electrical power that is required to activate the components in the hybrid analyte test meter. In this embodiment, a user activates the hybrid analyte test meter 104 via a user interface such a graphical icon or other input that the mobile electronic device 140 presents to the user as part of the user interface 172 in the application software 168. In one configuration, the mobile electronic device 140 continues to transmit electrical power to the hybrid meter 104 during operation of the hybrid meter 104, while in another embodiment the capacitor 132 receives sufficient charge prior to the operation of the hybrid meter 104 to analyze a single fluid sample that is applied to the test strip. The charging process typically enables the hybrid meter 104 to generate signal measurement data for a single fluid sample, and the mobile electronic device 140 provides additional electrical energy for each testing operation.

Referring to the mobile electronic device 140 in more detail, FIG. 2 depicts a second processor 144 that is operatively connected to a second memory 148, a short range wireless transceiver 152, input and output devices 156, and a wireless network transceiver 160. A battery 164, such as a lithium-ion battery or other suitable battery, provides electrical power to operate the processor 144, memory 148, a second short range wireless transceiver 152, input and output devices 156, wireless network transceiver 160, and, as described above, in some embodiments the battery 164 provides electrical power to the hybrid meter 104 via the second short range wireless transceiver 152. Since the mobile electronic device 140 is typically a general-purpose digital electronic device such as a smartphone, tablet, or wearable device, the mobile electronic device 140 includes commercially-available hardware components and the precise configuration of the mobile electronic device 140 vary based on manufacture. In general, the processor 144 is a system on a chip (SoC) that includes a CPU with one or more cores and a GPU that provides graphical output via the display device 158 of FIG. 1 or other graphical display devices. The processor 144 optionally includes digital signal processors for audio input and output and other specialized compute units including, for example, image processors and neural network accelerators. Other components in the mobile electronic device 140 including sensors such as accelerometers, gyroscopes, temperature sensors, humidity sensors, and the like are either integrated with the processor 144 or are operatively connected to the processor 144 and are described as being part of the processor 144 herein.

In the mobile electronic device 140, the memory 148 includes one or more non-volatile and volatile data storage devices. In the configuration of FIG. 2, the memory 148 stores application software 168 and operating system software 188 that both contain instructions for execution by the mobile electronic device processor 144.

The application software 168 further includes executable program code, configuration data, stored records of user preferences and a log of user data, and other digital assets such as graphical user interface (GUI) elements, for a user interface 172, analyte detection algorithm 176, communication stack 180, stored user data 184, and measurement signal data 186. In FIG. 1, the application software 168 is also depicted as including a copy of the firmware 118 that is used by the hybrid analyte test meter 104, since the mobile electronic device 140 receives the firmware 118 as part of a software update process that is described in further detail below. The user interface 172 includes software instructions and other graphical assets that enable the application software 168 to receive input from the user to control the analyte test meter 100 and to display the results of analyte tests along with other health related information for the user. The analyte detection algorithm 176 includes software instructions and profile and parameter data that enable the processor 144 to process the measurement signal data 186 that are received from the hybrid meter 104 to generate a measurement of the analyte level in a fluid sample. In some embodiments, analyte detection algorithm 176 also enables the processor 144 to execute failsafe protocols that detect contamination in the fluid sample or faults in the test strip. The communication software 180 interfaces with services provided by the operating system software 188 to send and receive data using both the short range wireless transceiver 152 that provides communication to the hybrid meter 104 and the wireless network transceiver 160 that enables the mobile electronic device 140 to send measurements of analyte levels to the healthcare services 288 for additional analysis. The stored user data 184 includes user-specific preference and configuration data as well as a history of one or more analyte measurements and optionally other information about the activities of the user include mealtime and activity data.

The operating system (OS) software 188 includes the software kernel, drivers, libraries, and other system software that are associated with a standard commercially-available operating system. The OS software 188 provides standardized services such as network and graphics stacks, filesystems for data storage and management, software access to the I/O devices 156, and the like. For explanatory purposes, the OS software 188 is also described herein as including other software applications beyond the application software 168 and, in particular, software update services that enable the mobile electronic device 140 to receive updates to the software 168 and the firmware 118 for the hybrid meter 104 from the software and firmware update services 284, even if these software programs are not strictly considered to be part of an “operating system” in an academic sense.

As described above, the processor 112 in the hybrid analyte test meter 104 executes stored program instructions in the firmware 118 and the processor 144 in the mobile electronic device 140 executes stored software instructions that implement the operating system software 188 and the application software 168. Of course, those of skill in the art recognize that the terms “firmware” and “software” both refer to stored program instructions and other data such as parameter data that are held in a non-transitory memory and that control the operation of a processor that executes the stored instructions. Both firmware and software may implement the functions described herein and both firmware and software may be updated during the operation of the hybrid analyte test meter 104 and the mobile electronic device 140. In the context of this disclosure, the terms “firmware” and “software” are used to provide a clear distinction between the operations of the hybrid analyte test meter 104 and the mobile electronic device 140, and these terms do not otherwise limit the scope of this disclosure.

In the mobile electronic device 140, the I/O devices 156 include, for example, input devices such as a touch-sensitive input in a display screen 158, mechanical buttons 159 or other mechanical control devices, voice input, haptic input, and the like. Output devices include, for example, graphical outputs such as the display screen 158, indicator lights, audio output via speakers or a headphone output, and the like.

In the mobile electronic device 140, the short range wireless transceiver 152 includes components that are compatible for communication with the short range wireless transceiver 128 in the hybrid meter 104, such as the aforementioned NFC transceiver or other short range wireless transceivers and antennas, such as a second coil antenna that enables wireless data communication and optionally electrical power transmission via inductive coupling between the mobile electronic device 140 and the hybrid meter 104. The wireless network transceiver 160 is a separate wireless device that communicates at longer ranges, such as on the order of several meters for Bluetooth or an IEEE 802.11 “Wi-Fi” connections and up to several kilometers for a cellular data transceiver, for communication with the software and firmware update services 284, the healthcare services 288, or other external computing systems via a network 280. Additionally the wireless network transceiver 160 is generally capable of communication with multiple devices including remote computing systems using an intermediary data network, such as the network 280 of FIG. 2, while the short range wireless transceivers 128 and 152 of FIG. 2 are generally configured for direct point-to-point communications between two devices, such as the hybrid analyte meter 104 and the mobile electronic device 140.

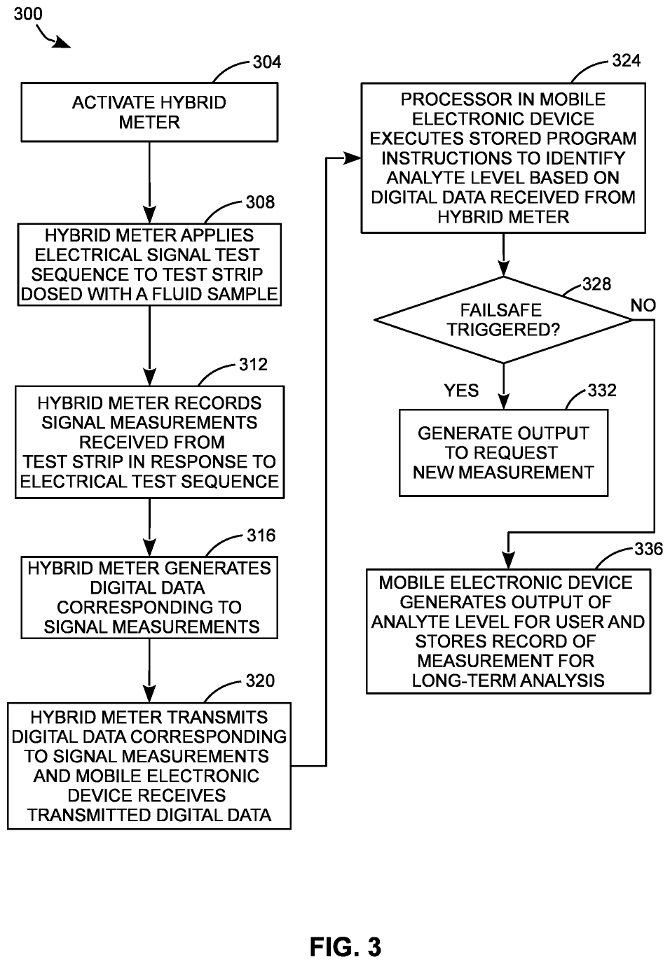

FIG. 3 depicts a process 300 for measurement of an analyte in a test sample using an analyte test meter that includes a hybrid analyte test meter and a mobile electronic device. In the description below, a reference to the process 300 performing a function or action refers to the operation of one or more processors in at least one of a hybrid analyte test meter or a mobile electronic device to execute stored program instructions to perform the function or action in conjunction with other components in an analyte test meter. The process 300 is described in conjunction with the analyte test meter 100 and the system 200 of FIG. 1 and FIG. 2 that implement an amperometric process to measure the level of a glucose analyte in a blood sample of a user who is a person with diabetes for illustrative purposes.

The process 300 begins with the activation of the hybrid meter 104 (block 304). In one configuration, a user executes the application software 168 by using a touchscreen, voice input, or other suitable input device 156 in the mobile electronic device 140. The processor 144 in the mobile electronic device 140 activates the short range wireless transceiver 152 to transmit an activation or “wakeup” signal to the corresponding short range wireless transceiver 128 in the hybrid meter 104. The processor 112 in the hybrid meter 104 activates from a deactivated or low-power standby operating mode in response to the activation signal. In an embodiment in which the hybrid meter 104 stores electrical energy in a capacitor 132 without using a battery, the activation signal from the mobile electronic device 140 charges the capacitor 132 to provide sufficient power to activate the hybrid analyte test meter 104. Additionally, if the user has not inserted a test strip into the port 108 the mobile electronic device 140 generates a graphical or audible output to instruct the user to insert the test strip as part of the activation process. In another configuration, the hybrid analyte test meter 104 activates upon insertion of a test strip into the port 108. The electrodes in the test strip close an electrical circuit to enable activation of the meter processor 112, which receives electrical power from a battery 132 or a previously charged capacitor 132. In this configuration, the meter processor 112 optionally transmits an activation signal to the mobile electronic device 140 using the short range wireless transceiver 128. The mobile electronic device processor 144 executes the application software 168 in response to receiving the activation signal without requiring additional input from the user beyond the insertion of the test strip. Alternatively, the user manually operates the mobile electronic device 140 to execute the application software 168.

The process 300 continues as the hybrid meter 104 applies an electrical signal test sequence to the test strip that has been dosed with a fluid sample (block 308). In the illustrative embodiment of FIG. 3, the electrical signal test sequence enables detection of a glucose level in a blood sample that is applied to the test strip. In one embodiment, the test sequence is a predetermined sequence of electrical signals including a plurality of alternating current (AC) signals followed by a plurality of direct current (DC) signals that the measurement signal generator 120 applies to the sample deposited on the electrochemical test strip via the port 108. For the detection of blood glucose in a blood sample, the measurement signal generator 120 generates an AC waveform followed by a series of pulsed DC signals that are applied to at least one circuit formed from a working electrode that is connected to a counter electrode via the chemical reagent that has received the dose of the fluid sample. While not described in further detail herein, the hybrid meter 104 optionally performs a dose sufficiency process that detects the application of the fluid sample, such as a blood sample, to the test strip by measuring levels of electrical impedance between one or more pairs of electrodes to ensure that the test strip has received a fluid sample prior to applying the electrical test signal sequence to the test strip.

In one configuration of predetermined sequence of AC and DC electrical signals, the measurement signal generator 120 generates AC signals with sinusoidal waveforms over a period of approximately 1.5 seconds with either a single frequency in a range of approximately 1 kHz to 100 kHz or a range of frequencies in various time segments, such as a series of time segments in which the measurement signal generator 120 generates AC signals with 10 kHz [in a first segment], 20 kHz, 10 kHz [in a second segment], 2 kHz, and 1 kHz frequencies, although other frequency progressions may be used as well. The measurement signal generator 120 generates the AC signal with a voltage amplitude of approximately ±0.05 V (0.1 V peak-to-peak amplitude), where this voltage level and other voltage levels in this example refer to a relative difference in potential between a working electrode and counter electrode in a test strip, such as the test strip 105, because this test strip does not include a separate reference electrode. The measurement signal generator 120 subsequently generates a series of pulsed DC signals with square or trapezoidal waveforms over a period of approximately 1.5 seconds. In one configuration, each DC pulse has a duration of approximately 100 milliseconds with a corresponding 100 millisecond relaxation period between pulse corresponding to a 50% duty cycle over a 200 millisecond period before commencing the next pulse, although the duration of each pulse may vary between, for example, 50 milliseconds to 500 milliseconds and the duty cycle may be greater than or less than 50%. The measurement signal generator 120 generates each DC pulse with a voltage of approximately 0.45 V, which is greater than the 0.1 V peak-to-peak amplitude of the AC signals. In this test sequence, the predetermined sequence of electrical signals including the AC and DC signals has a duration of approximately 3 seconds, and more generally the test sequences typically have a duration in a range of 1 to 10 seconds. The measurement signal generator 120 operates a switch to produce a short-circuit (0 V potential) between the electrodes in the test strip during each relaxation period and opens the switch during each DC pulse to ensure the DC pulse is applied in a circuit path that includes the reagent with the fluid sample.

The foregoing example is a non-limiting example of a predetermined sequence of electrical signals that are effective for the measurement of blood glucose values, and the frequencies, amplitudes, durations, and generation orders of the signals may be adjusted in alternative configurations. Additionally, alternative configurations can use a different sequence of AC and DC signals, or only use AC or DC signals to measure blood glucose or other analytes. For example, one alternative embodiment uses a DC pre-conditioning signal at the beginning of the predetermined sequence of electrical signals followed by the sequence described above or by only a pulsed-DC electrical signal sequence. Another alternative configuration generates a series of AC signals with triangular waveforms and higher voltage amplitudes (e.g. 0.45 V) after the series of pulsed DC signals, and the responses to these AC signals provide input data to one or more failsafe algorithms that detect potential faults in the test strip or contamination of the fluid sample that could prevent the accurate measurement of blood glucose.

During the process 300, the hybrid meter 104 records a plurality of signal measurements that are received from the test strip in response to the plurality of signals in the electrical test sequence (block 312) and generates digital data that correspond to the recorded signal measurements (block 316) for further processing using one or more digital logic devices. In the embodiments described herein, the analyte test meter 100 performs an amperometric analyte detection process to detect a glucose analyte in a blood sample. The measurement signal receiver 124 records signal measurements of electrical currents that are produced in the test strip in response to the predetermined sequence of electrical signals that the measurement signal generator 120 produces during the processing of block 308 that is described above. While the signal generator 120 generally operates with a predetermined voltage profile to generate the predetermined sequence of electrical signals in the test sequence, the measurement signal receiver 124 generally records the levels of electrical current that flow through the circuit formed by the electrodes and the dosed reagent in the test strip. These levels of electrical current are affected by the redox reaction between chemicals in the fluid sample, including the analyte, and the reagent that is formed on the test strip. Additionally, the electrical currents change over time based on changes in the electrical signals of the test sequence and the progression over time of the chemical reactions between the fluid sample and the reagent. Those of skill in the art will recognize that the measurement signal receiver 124 records signal measurements in response to the predetermined sequence of electrical signals both during and after the generate of signals by the measurement signal generator 120. For example, the measurement signal receiver 124 records electrical current measurements in response to the AC signals during the application of the AC signals, during the application of the pulsed DC signals, and during the relaxation period after each DC pulse during which the electrical current decays and, in some embodiments, temporarily reverses direction to produce a small negative current measurement through the circuit in the test strip for at least a portion of each relaxation period.

During the process 300, the measurement signal receiver 124 generates signal measurements by sampling the electrical current over time at the Nyquist rate of two or more times the highest frequency component of the signals in the test sequence, which is at least 40 kHz in the example described above, although the sampling rate may be higher or lower to maintain a sampling rate of at least two times the maximum frequency component of the test signals. Each signal measurement provides an analog measurement value of the current in a predetermined measurement range (e.g. a range of ±50 μA in one embodiment), and an analog to digital converter in the measurement signal receiver 124 or the hybrid meter processor 112 converts each analog signal measurement value to a digital data representation for further processing in the mobile electronic device 140. The hybrid meter processor 112 optionally buffers the digital data corresponding to the plurality of signal measurements in the meter memory 116 prior to transmission to the mobile electronic device 140, which is described in further detail below.

In addition to the digital value of each signal measurement, the hybrid meter processor 112 optionally generates a series of timestamp values and associates both the measurement signal data corresponding to the measured current responses and voltage level values corresponding to the voltages generated by the measurement signal generator 120 with the timestamp values. The timestamps and associated signal measurement and voltage value data enable the processor 144 in the mobile electronic device 140 to identify the temporal relationship between each signal measurement and the corresponding voltage signals that the measurement signal generator 124 applies to the test strip during the process 300. For example, these data enable detection of a phase difference between the electrical signals in the test sequence and the resulting measurement signals of the electrical current values that occur during at least the AC signal generation sequence that is described above. In another configuration, the hybrid meter 104 only transmits the data corresponding to the measurement signals to the mobile electronic device 140. In this configuration, the mobile electronic device 140 stores a time and voltage profile of the predetermined sequence of electrical signals in the test sequence as part of the analyte detection algorithm data 176. The processor 144 in the mobile electronic device associates the profile data with the corresponding signal measurement values based on the order in which the hybrid meter 104 generates the data corresponding to the signal measurement samples during the test sequence.

The process 300 continues as the hybrid meter 104 transmits the digital data corresponding to the plurality of signal measurements and optionally the timestamp and voltage values of the electrical signals in the test sequence to the mobile electronic device 140 (block 320). In the analyte test meter 100, the hybrid meter processor 112 operates the short range wireless transceiver 128 to transmit the data to the corresponding short range wireless transceiver 152 in the mobile electronic device. In one configuration, the hybrid analyte test meter 104 temporarily stores the digital data corresponding to the signal measurements in the meter memory 116 and transmits the stored digital data upon completion of recording digital representations of all the signal measurement data during the process 300. In another configuration, the hybrid analyte test meter 104 commences transmission of the digital representations of all the signal measurement data after at least one digital datum has been generated but prior to the completion of recording the digital data for the entire test sequence. The processor 144 in the mobile electronic device 140 temporarily stores the digital measurement signal data 186 in the memory 148 for additional processing to detect the level of the analyte in the fluid sample. The mobile electronic device 140 also stores timestamp and signal voltage data with the digital measurement signal data 186 in an embodiment in which the hybrid meter 104 transmits these data to the mobile electronic device 140.

The process 300 continues as the processor 144 in the mobile electronic device 140 executes stored program instructions of the analyte detection algorithms 176 in the application software 168 to generate a measurement of the analyte level based on the digital measurement signal data that the mobile electronic device 140 has received from the hybrid meter 104 (block 324). The measurement of the analyte level includes a general measurement based on the current measured during one or more of the DC pulses, identification and correction of confounding factors such as temperature and hematocrit levels in the blood sample that are corrected to improve the accuracy of the analyte measurement, and potentially the triggering of a failsafe if the processor 144 detects contamination of the fluid sample or a fault in the test strip. While algorithms that perform the analyte measurement process are known to the art and these algorithms are not described in full detail herein, in general the measured electrical current levels of the signal measurement data in response to the pulsed DC signals are positively correlated with the level of glucose in the sample, which enables the analyte detection algorithm to generate a measurement of the glucose level using a predetermined profile that maps the digital value of one or more of the signal measurements to a glucose level. While the level of glucose in the blood sample affects the current level in the signal measurements, other factors including temperature and the level of hematocrit in the blood sample also affect the current level in the signal measurements, and these variables are referred to as “confounding factors.” The meter processor 144 identifies characteristics of the signal measurement data, such as the phase differences between the generation of the AC voltage signals in the predetermined signal sequence and the corresponding current responses, to serve as inputs to correction functions in the analyte detection algorithm 176 that reduce or eliminate the spurious effects of the temperature and hematocrit variables, which increases the accuracy of the final blood glucose measurement.

During the process 300, the mobile electronic device processor 144 also performs failsafe detection as part of the analyte detection algorithm 176. While the analyte detection algorithm 176 corrects the confounding factors to improve the accuracy of the glucose level measurement, the failsafe functions of the analyte detection algorithm 176 detects certain external factors that preclude accurate detection of the glucose level. One example of the failsafe is the detection of the presence of elevated antioxidant levels in the blood sample, where ascorbic acid (vitamin C) is one example of an antioxidant that may contaminate the blood sample, which produces inaccurate glucose measurement results. Another failsafe occurs if one or more of the electrodes in the test strip have been damaged, which can result in either the detection of no current or interruptions in the flow of current in situations where the damaged electrodes only have intermittent electrical continuity. In the event of the triggering of a failsafe (block 328), the meter processor 144 does not generate a final glucose measurement. Instead, the meter processor 144 operates the user interface 172 in the application software 168 to generate a visual or audible output using the output devices 156 to alert the user to a failed test and to request a new measurement (block 332). In one embodiment, the output instructs the user to wash his or hands to reduce the likelihood of contamination and to replace the test strip prior to repeating the process 300 with a new test strip.

If the analyte measurement process completes without triggering a failsafe (block 328) then the process 300 continues as the processor 144 in the mobile device 140 generates an output with the output device to present the level of the analyte in the sample to a user and optionally stores a record of the measurement with the stored user data 184 for long-term analysis (block 336). In one embodiment, the meter processor 144 operates the user interface 172 in the application software 168 to generate an visual or audible output of a numeric measurement of the measured glucose level (e.g. in units of milligrams of glucose per deciliter of blood such as 100 mg/dL) via the display device 158. In addition to the numeric output, the mobile electronic device 140 optionally generates an additional output with a history of blood glucose measurements or advice for managing blood glucose levels if the measured glucose level lies above or below a suggested range.

In the system 200, the analyte test meter 100 stores the measured blood glucose level for the user in association with the stored user data 184 in the mobile electronic device memory 148. The memory 148 stores the blood glucose measurement in association with the date and time at which the measurement was generated. In addition to storage in the memory 148, the mobile electronic device 140 optionally executes the communication software 180 to transmit the glucose measurement and associated user data to the healthcare services system 288 using the wireless network transceiver 160 for long-term storage and additional analysis. The associated user data can include other information from the user, such as a manual input that indicates the most recent time that the user consumed food prior to the blood glucose measurement, and automated data such as the location of the mobile electronic device 140 at the time of the blood glucose measurement, and accelerometer data that may indicate the level of activity of the user prior to taking the blood glucose measurement. As described above, the healthcare services system 288 performs additional analysis of long-term trends in the blood glucose levels and other health parameters of the user and each blood glucose measurement provides additional input data to the healthcare services 288. The transmission of the blood glucose level to the healthcare services 288 occurs without requiring manual input on the part of the user, which enables efficient automated tracking of the blood glucose levels of the user over time for review by both the user and authorized healthcare providers. Upon completion of the process 300, the user removes and disposes of the test strip 105, and the hybrid analyte test meter 104 optionally includes a mechanical ejection mechanism to facilitate removal of the test strip 105. The hybrid analyte test meter 104 deactivates until the user commences the process 300 again, and the processor 144 in the mobile electronic device 140 can execute other software applications in the operating system software 188 without requiring disconnection of the hybrid analyte test meter 104. The hybrid analyte test meter 104 remains affixed to the mobile electronic device 140 in the case 126 and does not interfere with the operation of the mobile electronic device 140 for general-purpose use when the hybrid analyte test meter 104 is deactivated.

As described above, the analyte test meter 100 incorporates the hybrid meter 104 and a specifically reconfigured mobile electronic device 140 to enable the measurement of an analyte in a fluid sample, such as the measurement of glucose in a blood sample. In addition to the advantages over prior art analyte measurement devices that are described above, the analyte test meter 100 further enables dynamic software and firmware updates that enable dynamic modification and improvement to the operation of the analyte test meter 100 without requiring the replacement of the hybrid meter 104 or the mobile electronic device 140.

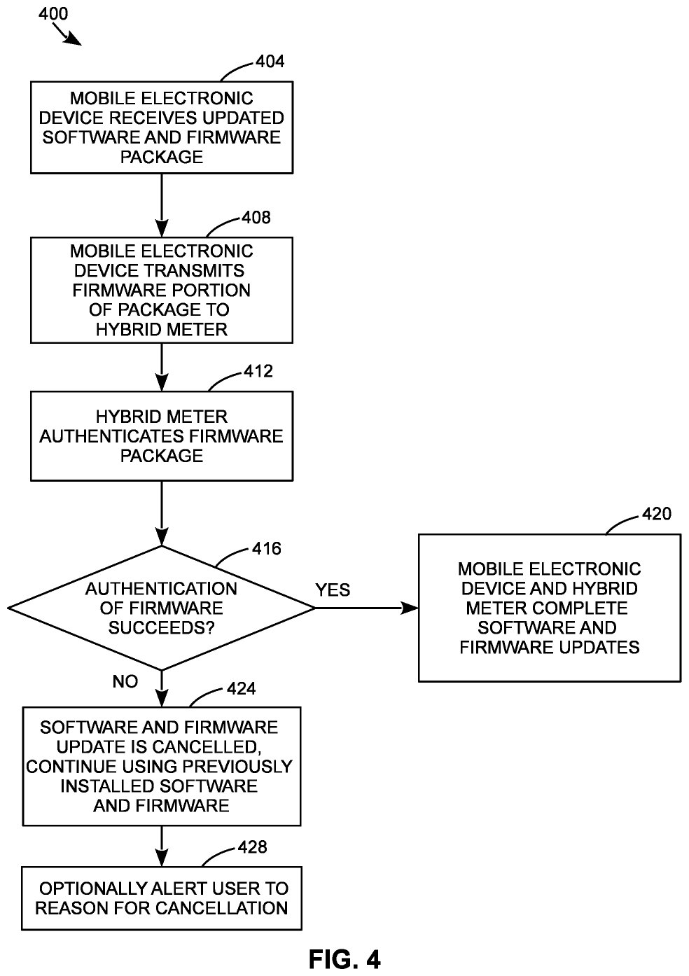

FIG. 4 depicts a process 400 for the operation of the analyte meter 100 to update either or both of the application software 168 in the mobile electronic device 140 and the firmware 118 in the hybrid meter 104. In the description below, a reference to the process 400 performing a function or action refers to the operation of one or more processors in at least one of a hybrid analyte test meter or a mobile electronic device to execute stored program instructions to perform the function or action in conjunction with other components in an analyte test meter. The process 400 is described in conjunction with the analyte test meter 100 and the system 200 of FIG. 1 and FIG. 2 for illustrative purposes.

The process 400 begins as the mobile electronic device 140 receives an updated software package from the online software and update services 284 (block 404). In the system 200, the processor 144 in the mobile electronic device 140 executes a software update program in the operating system software 188 that uses the wireless network transceiver 160 to retrieve the software package from the online software and update services 284 via the network 280. The mobile electronic device 140 optionally uses a software update service that is also referred to as an “app store” that provides software updates for mobile electronic devices. In one configuration, the software package includes an updated replacement for the executable software instruction code for the analysis software 168, configuration file data including parameter data that the application software uses during the analyte detection process, various graphical assets that are used to generate the user interface, and additional firmware code that the mobile electronic device 140 receives for subsequent transfer to the hybrid meter 104. The processor 144 in the mobile electronic device 140 extracts the files and other data structures from the software package and stores them in the memory 148 as part of the process 400, although a copy of a previously installed version of the application software 168 remains in the memory in case the update process fails during a firmware update operation that is described in more detail below.

In another configuration in which the mobile electronic device 140 and the hybrid meter 104 are already configured with a prior version of the application software 168 and the firmware 118, the software package only includes a set of files that include changes to the existing software 168 and firmware 118 while other portions of the software remain unchanged. For example, one type of update includes changes to the parameters used in the glucose detection algorithm or in the failsafe detection process, but does not affect the executable instructions in either the application software 168 or the firmware 118, and the software package only contains the relevant changes to the parameter data without requiring a full replacement of the entire software and firmware.

The process 400 continues as the mobile electronic device 140 transmits updated firmware data to the hybrid analyte test meter 104 (block 408). The software package includes a copy of the firmware 118, and the processor 144 operates the short range wireless transceiver 152 to transmit the firmware 118 to the hybrid meter 104, where the processor 112 in the hybrid meter 104 stores the firmware data in the meter memory 116. The meter memory 116 include sufficient capacity to store at least two copies of the firmware data 118, which enables the hybrid meter 104 to retain use of the existing firmware 118 until the updated firmware has been fully verified prior to using the updated firmware. As described above, in some configurations a software update may not include updates to the firmware for the hybrid analyte test meter 104, so the firmware update process may not occur during every software update, but the analyte test meter 100 is configured to perform the firmware update when an update is included as part of the software update.

The process 400 continues as the hybrid meter 104 authenticates the firmware data that have been received from the mobile electronic device 140 (block 412). In the authentication process, the meter processor 112 uses the authentication key 119, which is stored in the meter memory 116 at the time of manufacture and is separate from the firmware data 118, to verify the authenticity of the firmware data that are received from the mobile electronic device 140 based on a cryptographic signature that the mobile electronic device 140 receives from the software and firmware update services 284 either as part of the updated firmware data or in conjunction with the updated firmware data, and the hybrid analyte test meter 104 also stores the cryptographic signature in the meter memory 116.

In one embodiment, the authentication key 119 is a public key that is associated with a private key that is known only to an authorized party, such as the device manufacturer of the hybrid analyte test meter 104 that has received regulatory approval to distribute an version of the firmware. A computing system of the authorized performs a signing operation that generates a cryptographic hash value of the updated firmware data using, for example, the SHA-3 or other suitable cryptographically-secure hash algorithm, and then produces the cryptographic signature by using a private key to encrypt the hash value using an asymmetric cryptographic algorithm for later decryption using the public authentication key 119. The private key is not disclosed to the analyte test device 100 or to any other computing systems in the system 200, although the authentication key 119 in the memory 116 of the hybrid meter 104 need not be a secret and can be made publicly known. Those of skill in the art will of course recognize that the encryption described in the context of a cryptographic signature and authentication process does not make the SHA-3 value in the signature a secret because the publicly-available authentication key 119 can decrypt this value. Instead, the meter processor 112 uses the authentication key 119 to decrypt the cryptographic hash value that can only be generated using the corresponding private signing key and cannot be forged in a practical manner. The meter processor 112 also uses the same cryptographically-secure hash algorithm that was used to generate the decrypted cryptographic hash value (e.g. SHA-3) to generate another cryptographic hash value of the updated firmware data. The meter processor 112 compares the calculated cryptographic hash value of the updated firmware data to the decrypted cryptographic hash value from the signature. If the processor 112 verifies that the two values match, then the meter processor 112 successfully authenticates the digital signature and the firmware data because only the party with the private key can practically generate the signature that matches the cryptographic hash value for the firmware data. If, however, the processor 112 verifies that the cryptographic hash value does not match the cryptographic hash value that is decrypted from the cryptographic signature, then the authentication process fails because the hybrid meter 104 has confirmed that either or both of the received firmware data and the cryptographic signature are not authentic.

During the process 400, even if the software and firmware update services 284 are not under the direct control of the device manufacturer, and this is expected to be the case in most practical embodiments of the system 200, the operators of the software and firmware update services 284 or a malicious third-party cannot effectively modify the firmware 118 in a manner that avoids detection by the hybrid analyte test meter 104, which prevents an unauthorized party from loading non-approved firmware in the hybrid meter 104. In some embodiments, each firmware image includes a version number, which is part of the signed firmware data and cannot be altered without detection, and the meter processor 112 also confirms that the version of the updated firmware is newer (e.g. a larger version number) than the currently installed firmware. This prevents an unauthorized third-party from successfully loading an otherwise valid but outdated firmware even if the outdated firmware has a valid signature. Furthermore, the authorization process also ensures that the firmware received from the mobile electronic device 140 has not been corrupted due to data transmission or other hardware errors.

Referring again to FIG. 4, if the authentication of the firmware succeeds (block 416), then the hybrid meter 104 and the mobile electronic device 140 complete the software update process (block 420). In one embodiment, the meter processor 112 either deactivates the hybrid meter 104 to await a future activation during the process 300 or reboots immediately and uses the newly updated firmware data that are stored in the hybrid meter memory 116 as the new meter firmware 118. The meter processor 112 optionally deletes the old firmware data from the memory 116 after a successful update. The meter processor 112 also transmits a message to the mobile electronic device 140 using the short range wireless transceiver to indicate that the authentication was successful either prior-to or subsequently-to the completion of the firmware updated process. In the mobile electronic device 140, the processor 144 completes the update of the application software data to a new version in response to receiving the message from the hybrid meter 104 indicating that the firmware has been successfully updated. The mobile electronic device processor 144 typically restarts the application software program 168 to use the updated version and optionally deletes the older version of the application data 168 from the mobile electronic device memory 148. While not described in further detail herein, the mobile electronic device 140 optionally authenticates the validity of components in the application software 168 using additional cryptographic signatures in a similar manner to the firmware authentication that is described above, although the mobile electronic device 140 optionally uses a more complex process involving chained-certificates that are authenticated based on trusted public keys from certificate authorities in a manner that is otherwise known to the art in systems such as the transaction-layer security (TLS) protocol. Upon completion of the updates to the application software 168 and the firmware 118, the analyte test meter 100 can perform the process 300 and other functions using newly updated software.

Referring again to FIG. 4, during the process 400, if the authentication of the firmware does not succeed (block 416), then the software and firmware update process is cancelled (block 424). In the hybrid meter 104, the meter processor 112 uses the short range wireless transceiver 128 to transmit a message to the mobile electronic device 140 indicating failure of the authentication process. The meter processor 112 continues to use the previous version of the firmware 118, and optionally deletes the firmware data and cryptographic signature that failed the authentication process from the meter memory 116. The mobile electronic device processor 144 also continues to use the prior version of the application software 168 and does not execute the updated version of the application software data. The mobile electronic device processor 144 optionally deletes the updated application software data from the memory 148 in response to a failure in the authentication process. The mobile electronic device processor 144 optionally generates an error output message to inform the user of the reason for the failure in the software update due to the authentication failure via the user interface 172 and the I/O devices 156 (block 428).

As described above, the process 400 enables dynamic updates to both the application software 168 in the mobile electronic device 140 and the firmware 118 in the hybrid analyte test meter 104. A non-limiting list of technical advantages provided over the prior art that are provided by the embodiments described herein include the capability to change the parameters used for analyte detection and for the detection of failsafe conditions, to change the predetermined sequence of electrical signals that the hybrid meter 104 applies to the test strip during the test sequence, and to change the analyte detection and failsafe algorithms that the mobile electronic device 140 uses to detect the analyte levels and trigger failsafes if necessary.

This disclosure is described in connection with what are considered to be the most practical and preferred embodiments. However, these embodiments are presented by way of illustration and are not intended to be limited to the disclosed embodiments. Accordingly, one of skill in the art will realize that this disclosure encompasses all modifications and alternative arrangements within the spirit and scope of the disclosure and as set forth in the following claims.

What is claimed is:

1. A hybrid analyte test meter comprising:
a memory configured to store firmware instructions;
a port configured to receive an electrochemical test strip;
a measurement signal generator electrically connected to the port;
a measurement signal receiver electrically connected to the port;
a short range wireless transceiver; and
a processor operatively connected to the memory, the measurement signal generator, the measurement signal receiver, and the short range wireless transceiver, the processor being configured to execute the firmware instructions in the memory to:
operate the measurement signal generator to apply a predetermined sequence of electrical signals to a sample deposited on the electrochemical test strip via the port;
receive a plurality of signal measurements from the measurement signal receiver, the measurement signal receiver generating the plurality of measured signals based on a plurality of electrical signals received from the electrochemical test strip in the port in response to the predetermined sequence of electrical signals; and
transmit data corresponding to the plurality of signal measurements to an external computing device using the short range wireless transceiver, wherein the data corresponding to the plurality of signal measurements enable another processor in the external computing device to identify a measurement of an analyte in the sample.

2. The hybrid analyte test meter of claim 1, the short range wireless transceiver further comprising:
a near field communication (NFC) transceiver.

3. The hybrid analyte test meter of claim 1 further comprising:
a capacitor configured to provide electrical power to the memory, the measurement signal generator, the measurement signal receiver, the short range wireless transceiver, and the processor; and
a coil antenna electrically connected to the short range wireless transceiver and the capacitor, wherein the coil antenna is configured to inductively couple with another coil antenna in the external computing device to charge the capacitor with electrical energy received from the external computing device.

4. The hybrid analyte test meter of claim 3, wherein the memory, the measurement signal generator, the measurement signal receiver, and the processor are activated in response to the capacitor reaching a predetermined charge level.

5. The hybrid analyte test meter of claim 1 further comprising:
a battery configured to provide electrical power to the memory, the measurement signal generator, the measurement signal receiver, the short range wireless transceiver, and the processor, wherein the memory, the measurement signal generator, the measurement signal receiver, the short range wireless transceiver, and the processor are activated using electrical energy received from the battery in response to an insertion of the electrochemical test strip into the port.

6. The hybrid analyte test meter of claim 1, the memory being further configured to store:
an authentication key; and
the processor being further configured to execute the firmware instructions in the memory to:
receive updated firmware data and a cryptographic signature corresponding to the updated firmware data from the external computing device using the short range wireless transceiver;
store the updated firmware data and the cryptographic signature in the memory;
generate a hash value of the updated firmware data using a cryptographically-secure hash function; and
execute stored instructions in the updated firmware data only in response to verification that the hash value matches an output of a decryption of the cryptographic signature using the authentication key.

7. The hybrid analyte test meter of claim 6, the processor being further configured to execute the firmware instructions in the memory to:
delete the updated firmware data from the memory in response to verification that the hash value does not match the output of a decryption of the cryptographic signature using the authentication key.

8. The hybrid analyte test meter of claim 1, the processor being further configured to execute the firmware instructions in the memory to:
operate the measurement signal generator to apply the predetermined sequence of electrical signals comprising a plurality of alternating current (AC) signals followed by a plurality of direct current (DC) signals to the sample deposited on the electrochemical test strip via the port.

9. The hybrid analyte test meter of claim 1 further comprising:
a container configured to store a plurality of the electrochemical test strips.

10. An analyte test meter comprising:
a hybrid analyte test meter and a mobile electronic device, the hybrid analyte test meter comprising:
a first memory configured to store firmware instructions;
a port configured to receive an electrochemical test strip;
a measurement signal generator electrically connected to the port;
a measurement signal receiver electrically connected to the port;
a first short range wireless transceiver; and
a first processor operatively connected to the first memory, the measurement signal generator, the measurement signal receiver, and the first short range wireless transceiver, the first processor being configured to execute the firmware instructions in the first memory to:
operate the measurement signal generator to apply a predetermined sequence of electrical signals to a sample deposited on the electrochemical test strip via the port;
receive a plurality of signal measurements from the measurement signal receiver, the measurement signal receiver generating the plurality of signal measurements based on a plurality of electrical signals received from the electrochemical test strip in the port in response to the predetermined sequence of electrical signals; and
transmit data corresponding to the plurality of signal measurements to the mobile electronic device using the first short range wireless transceiver; and
the mobile electronic device comprising:
a second memory configured to store software instructions;
a second short range wireless transceiver;
an output device; and
a second processor operatively connected to the second memory, the second short range wireless transceiver, and the output device, the second processor being configured to execute the software instructions in the second memory to:
receive the plurality of signal measurements from the hybrid analyte test meter using the second short range wireless transceiver;
execute an analyte detection algorithm to identify a level of the analyte in the sample based on the plurality of signal measurements; and
generate an output with the output device to present the level of the analyte in the sample to a user.

11. The analyte test meter of claim 10, wherein the first short range wireless transceiver is a first near field communication (NFC) transceiver and the second short range wireless transceiver is a second NFC transceiver.

12. The analyte test meter of claim 10, the hybrid analyte test meter further comprising:
a capacitor configured to provide electrical power to the first memory, the measurement signal generator, the measurement signal receiver, the first short range wireless transceiver, and the first processor; and
a first coil antenna electrically connected to the first short range wireless transceiver and the capacitor; and
the mobile electronic device further comprising:
a battery; and
a second coil antenna electrically connected to the second short range wireless transceiver and the battery, wherein the first coil antenna in the hybrid analyte test meter is configured to inductively couple with the second coil antenna in the mobile electronic device to charge the capacitor with electrical energy received from the battery in the mobile electronic device.

13. The analyte test meter of claim 12, the mobile electronic device further comprising:
an input device; and
the second processor being operatively connected to the input device and further configured to execute the software instructions in the second memory to:
receive an input request to activate the hybrid analyte test meter using the input device; and
activate the second short range wireless transceiver to transfer electrical energy from the battery to the capacitor in the hybrid analyte test meter to charge the capacitor to a predetermined level for activation of the hybrid analyte test meter.

14. The analyte test meter of claim 12, wherein the first memory, the measurement signal generator, the measurement signal receiver, and the first processor in the hybrid analyte test meter are activated in response to the capacitor reaching a predetermined charge level.

15. The analyte test meter of claim 12, wherein the second processor is configured to execute the analyte detection algorithm to identify a level of glucose in a blood sample based on the plurality of signal measurements.

16. The analyte test meter of claim 10, the hybrid analyte test meter further comprising:
a battery configured to provide electrical power to the memory, the measurement signal generator, the measurement signal receiver, the short range wireless transceiver, and the processor, wherein the memory, the measurement signal generator, the measurement signal receiver, the short range wireless transceiver, and the processor are activated using electrical energy received from the battery in response to an insertion of the electrochemical test strip into the port.

17. The analyte test meter of claim 10, the first memory in the hybrid analyte test meter being further configured to store:
an authentication key; and
the first processor in the hybrid analyte test meter being further configured to execute the firmware instructions in the memory to:
receive updated firmware data and a cryptographic signature corresponding to the updated firmware data from the external computing device using the first short range wireless transceiver;
store the updated firmware data and the cryptographic signature in the first memory;
generate a hash value of the updated firmware data using a cryptographically-secure hash function; and
execute stored instructions in the updated firmware data only in response to verification that the hash value matches an output of a decryption of the cryptographic signature using the authentication key.

18. The analyte test meter of claim 17, the first processor in the hybrid analyte test meter being further configured to execute the firmware instructions in the memory to:
delete the updated firmware data from the memory in response to verification that the hash value does not match the output of a decryption of the cryptographic signature using the authentication key.

19. The analyte test meter of claim 10, the first processor in the hybrid analyte test meter being further configured to execute the firmware instructions in the memory to:
operate the measurement signal generator to apply the predetermined sequence of electrical signals comprising a plurality of alternating current (AC) signals followed by a plurality of direct current (DC) signals to the sample deposited on the electrochemical test strip via the port.

20. The analyte test meter of claim 10, the hybrid analyte test meter further comprising:
a container configured to store a plurality of the electrochemical test strips.

21. The analyte test meter of claim 10 further comprising:
a case comprising a first cavity that contains the hybrid analyte test meter and a second cavity that contains the mobile electronic device, wherein the case holds the hybrid analyte test meter in place proximate to a rear surface of the mobile electronic device.
